(19) United States
(12) Patent Application Publication
(10) Pub. No.: US 2024/0038370 A1
(43) Pub. Date: Feb. 1, 2024

(54) TREATMENT OUTCOME PREDICTION FOR NEOVASCULAR AGE-RELATED MACULAR DEGENERATION USING BASELINE CHARACTERISTICS

(71) Applicant: Genentech, Inc., South San Francisco, CA (US)

(72) Inventors: Neha Sutheekshna ANEGONDI, Fremont, CA (US); Jian DAI, Fremont, CA (US); Michael Gregg KAWCZYNSKI, Nevada City, CA (US); Yusuke Alexander KIKUCHI, South San Francisco, CA (US)

(21) Appl. No.: 18/482,237

(22) Filed: Oct. 6, 2023

Related U.S. Application Data
(63) Continuation of application No. PCT/US2022/023931, filed on Apr. 7, 2022.
(60) Provisional application No. 63/172,063, filed on Apr. 7, 2021.

(51) Int. Cl. G16H 30/40; G16H 10/60; G06T 7/00

(52) U.S. Cl. CPC G16H 30/40; G16H 10/60; G06T 7/0012; G06T 2207/30041; G06T 2207/10101; G06T 2207/20081

(57) ABSTRACT

A method and system for predicting a treatment outcome. Three-dimensional imaging data for a retina of a subject is received. A first output is generated using a deep learning system and the three-dimensional imaging data. The first output and baseline data are received as input for a symbolic model. A treatment outcome is predicted, via the symbolic model, for the subject undergoing a treatment for neovascular age-related macular degeneration (nAMD) using the input.

### CROSS-REFERENCE TO RELATED APPLICATIONS

This application is a continuation of International Application No. PCT/US2022/023931, filed Apr. 7, 2022, and entitled “Treatment Outcome Prediction For Neovascular Age-Related Macular Degeneration Using Baseline Characteristics,” which claims priority to U.S. Provisional Patent Application No. 63/172,063, filed Apr. 7, 2021, and entitled “Treatment Outcome Prediction for Neovascular Age-Related Macular Degeneration using Baseline Characteristics,” which are incorporated herein by reference in their entirety.

### FIELD

This description is generally directed towards predicting treatment outcomes in subjects diagnosed with age-related macular degeneration. More specifically, this description provides methods and systems for predicting treatment outcomes in subjects diagnosed with neovascular age-related macular degeneration (nAMD) using baseline data identified for the subjects.

### BACKGROUND

Age-related macular degeneration (AMD) is a disease that impacts the central area of the retina in the eye, which is referred to as the macula. AMD is a leading cause of vision loss in subjects 50 years or older. Neovascular AMD (nAMD) is one of the two advanced stages of AMD. With nAMD, new and abnormal blood vessels grow uncontrollably under the macula. This type of growth may cause swelling, bleeding, fibrosis, other issues, or a combination thereof. The treatment of nAMD typically involves an anti-vascular endothelial growth factor (anti-VEGF) therapy (e.g., an anti-VEGF drug such as ranibizumab). The retina's response to such treatment is at least partially subject specific, such that different subjects may respond differently to the same type of anti-VEGF drug. Further, anti-VEGF therapies are typically administered via intravitreal injections, which can be expensive and themselves cause complications (e.g., blindness).

### SUMMARY

In one or more embodiments, a method for predicting a treatment outcome is provided. Three-dimensional imaging data for a retina of a subject is received. A first output is generated using a deep learning system and the three-dimensional imaging data. The first output and baseline data are received as input for a symbolic model. A treatment outcome is predicted, via the symbolic model, for the subject undergoing a treatment for neovascular age-related macular degeneration (nAMD) using the input.

In one or more embodiments, a method for predicting a treatment outcome for a subject undergoing a treatment for neovascular age-related macular degeneration (nAMD). A first predicted outcome is generated using a deep learning system and three-dimensional imaging data for a retina of the subject. A second predicted outcome is generated using a symbolic model and baseline data for the subject. The treatment outcome is predicted for the subject undergoing the treatment for nAMD using the first predicted outcome and the second predicted outcome.

In one or more embodiments, a system for managing an anti-vascular endothelial growth factor (anti-VEGF) treatment for a subject diagnosed with neovascular age-related macular degeneration (nAMD) comprises a memory containing machine readable medium comprising machine executable code and a processor coupled to the memory. The processor configured to execute the machine executable code to cause the processor to: receive three-dimensional imaging data for a retina of a subject; generate a first output using a deep learning system and the three-dimensional imaging data; receive the first output and baseline data as input for a symbolic model; and predict, via the symbolic model, a treatment outcome for the subject undergoing a treatment for neovascular age-related macular degeneration (nAMD) using the input.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a prediction system in accordance with various embodiments.

FIG. 2 is a flowchart of a process for predicting a treatment outcome in accordance with various embodiments.

FIG. 3 is a flowchart of a process for predicting a treatment outcome in accordance with various embodiments.

FIG. 4 is a flowchart of a process for predicting a treatment outcome in accordance with various embodiments.

FIG. 5 is a table showing the performance data for a model stacking and model averaging approach in predicting a treatment outcome in accordance with one or more embodiments.

FIG. 6 is a table showing the performance data for a model stacking and model averaging approach in predicting a treatment outcome in accordance with one or more embodiments.

FIG. 7 is a block diagram of a computer system in accordance with one or more embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

Determining a subject's response to an age-related. macular degeneration (AMD) treatment and, in many cases, in particular, to a neovascular AMD (nAMD) treatment, may include determining the subjects visual acuity response, the subject's reduction in fovea! thickness, or both. A subject's visual acuity may be the sharpness of his or her vision, which may be measured by the subject's ability to discern letters or numbers at a given distance. Visual acuity is oftentimes ascertained via an eye exam and measured according to the standard Snellen eye chart. Retinal images may provide information that can be used to estimate a subject's visual acuity. For example, optical coherence tomography (OCT) images nay be used to estimate a subject's visual acuity at the time the OCT images were captured. Foveal thickness, which is also referred to as central subfield thickness (CST), may be defined as the average thickness of the macula in the central 1 mm diameter area. CST may also be measured using OCT images.

But in certain cases, such as, for example, in clinical trials, being able to predict a subject's future response to an AMI) treatment (e.g., nAMD treatment) may be desirable. For example, it may be desirable to predict whether a subject's visual acuity will have improved at a selected period of time after treatment (e.g., at 6 months after treatment, 9 months after treatment, at 12 months after treatment, at 24 months after treatment, etc.). Further, it may be desirable to classify any such improvement in visual acuity. In some cases, it may be desirable to predict whether a subject will experience a reduction in CST (e.g., any reduction in CST or a reduction greater than a selected threshold). Such predictions and classification may enable treatment regimens to be personalized for a given subject. For example, predictions about a subject's visual acuity response to a particular AMD treatment may be used to customize the injection dosage, the intervals at which injections are given, or both. Further, such predictions may improve clinical trial screening, prescreening, or both by enabling; the exclusion of those subjects predicted to not respond well to treatment.

Thus, the various embodiments described herein provide methods and systems for predicting treatment outcomes for subjects in response to AMD treatment (e.g., nAMD treatment). In particular, baseline data is input into a symbolic model and used to predict an outcome for a subject undergoing such a treatment. The outcome may include, for example, without limitation, a predicted visual acuity measurement, a predicted change in visual acuity, a predicted central subfield thickness, a predicted reduction in central subfield thickness, or a combination thereof. In some embodiments, the input sent into the symbolic model includes both the baseline data and an output (e.g., a previously generated predicted outcome) generated based on three-dimensional imaging data (e.g., OCT imaging data). For example, OCT imaging data may be processed via a deep learning system to generate a predicted outcome that is combined with the baseline data. In this manner, the baseline data and this predicted outcome are fused to form an input that is sent into the symbolic model.

In other embodiments, the symbolic model may be used to generate a first output using the baseline data and the deep learning system is used to generate a second output using the three-dimensional imaging data. These two outputs are combined, fused, or otherwise integrated to form an outcome output that includes or indicates a predicted treatment outcome. For example, the first output and the second output may be a first predicted outcome and a second predicted outcome, respectively. A weighted average (e.g., equally weighted average) of these two predicted outcomes may be used as the final treatment outcome for a subject.

Recognizing and taking into account the importance and utility of a methodology and system that can provide the improvements described above, the embodiments described herein provide methods and systems for predicting visual acuity response to an AMD treatment (e.g., nAMD treatment). More particularly, the embodiments described herein provide methods and systems for processing baseline data using a symbolic model to predict treatment outcomes in subjects undergoing nAMD treatment at a selected period of time (e.g., 6 months, 9 months, 12 months, 24 months, etc.) after a baseline point in time. The baseline point in time may be, for example, but is not limited to, day one of treatment. Using the methods and systems described herein may have the technical effect of reducing the overall computing resources and/or time needed to predict treatment outcomes in subjects undergoing nAMD treatment. Further, using the methods and systems may allow treatment outcomes in subjects to be predicted more efficiently and accurately as compared to other methods and systems.

Moreover, the embodiments described herein may facilitate the creation of personalized treatment regimens for individual subjects to ensure the proper dosage and/or intervals between treatment doses (e.g., injections). In particular, the embodiments described herein may help generate accurate, efficient, and expedient personalized treatment or dosing schedules and enhance clinical cohort selection or clinical trial design.

### II. Prediction of Neovascular Age-Related Macular Degeneration (nAMD) Treatment Outcome

II.A. Exemplary Prediction System for Predicting AMD Treatment Outcomes

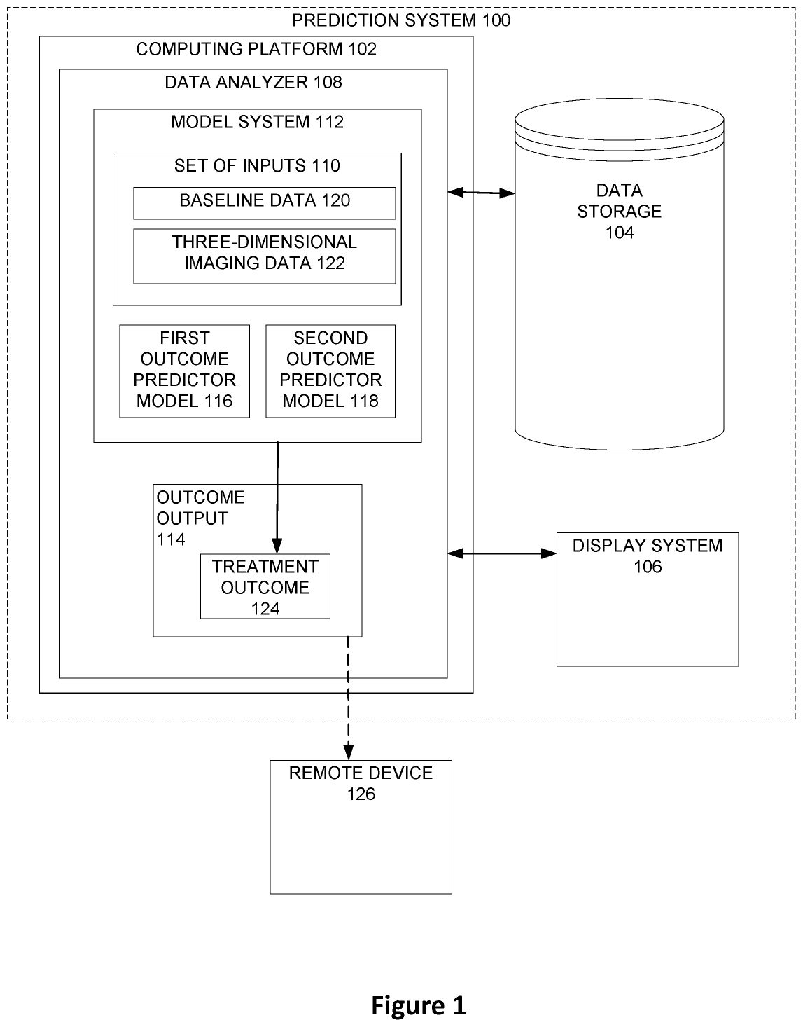

FIG. 1 is a block diagram of a prediction system 100 in accordance with various embodiments. Prediction system 100 is used to predict a treatment outcome for one or more subjects with respect to an AMD treatment. The AMD treatment, which may be an nAMD treatment, may include, for example, but is not limited to, an anti-VEGF treatment, an antibody treatment, another type of treatment, or a combination thereof. The anti-VEGF treatment may include, for example, ranibizumab, which may be administered via intravitreal injection. The antibody treatment may be, for example, a monoclonal antibody treatment that targets the vascular endothelial growth factor (VEGF) and angiopoietin 2 inhibitor. In one or more embodiments, the antibody treatment includes faricimab.

Prediction system 100 includes computing platform 102, data storage 104, and display system 106. Computing platform 102 may take various forms. In one or more embodiments, computing platform 102 includes a single computer (or computer system) or multiple computers in communication with each other. In other examples, computing platform 102 takes the form of a cloud computing platform. In some examples, computing platform 102 takes the form of a mobile computing platform (e.g., a smartphone, a tablet, a smartwatch, etc.).

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

Prediction system 100 includes data analyzer 108, which may be implemented using hardware, software, firmware, or a combination thereof. In one or more embodiments, data analyzer 108 is implemented in computing platform 102. Data analyzer 108 processes a set of inputs 110 using model system 112 to predict (or generate) outcome output 114.

Model system 112 may include any number of or combination of artificial intelligence models or machine learning models. In one or more embodiments, model system 112 includes a first outcome predictor model 116 and a second outcome predictor model 118. In one or more embodiments, first outcome predictor model 116 includes a deep learning system, which may include, for example, one or more neural networks, with at least one of these one or more neural networks being a deep learning neural network (or deep neural network) (DNN). In one or more embodiments, second outcome predictor model 118 includes a symbolic model, the symbolic model including one or more models that use symbolic learning or symbolic reasoning. For example, second outcome predictor model 118 may include, without limitation, at least one of a linear model, a random forest model, an Extreme Gradient Boosting (XGBoost) algorithm, or another type of model or algorithm.

In one or more embodiments, set of inputs 110 sent into model system 112 may be at least partially received from a source external to prediction system 100 over one or more communications links (e.g., wired communications links, wireless communications links, optical communications links, etc.). In one or more embodiments, set of inputs 110 is at least partially retrieved from data storage 104.

Set of inputs 110 for model system 112 may include, baseline data 120. In one or more embodiments, set of inputs 110 may additionally include three-dimensional imaging data 122. Baseline data 120 includes data obtained for a baseline point in time. The baseline point in time may be, for example, a point in time prior to treatment or a point in time concurrent with a first dose of a treatment (e.g., day one of treatment).

Baseline data 120 may include, for example, without limitation, at least one of demographic data, a baseline visual acuity measurement, a baseline CST measurement, a baseline low-luminance deficit (LLD), a treatment arm, or some other type of baseline measurement. The demographic data may include, for example, without limitation, at least one of age, gender, or another type of demographic metric. The baseline visual acuity measurement may be, for example, a best corrected visual acuity (BCVA) measurement. The baseline CST measurement may be, for example, in micrometers. The LLD may be the difference between a baseline BCVA measurement and a baseline low-luminance visual acuity (LLVA) measurement.

Three-dimensional imaging data 122 may include OCT imaging data, data extracted from OCT images (e.g., OCT en-face images), tabular data extracted from OCT images, some other form of imaging data, or a combination thereof. The OCT imaging data may include, for example, spectral domain OCT (SD-OCT) B-scans. Three-dimensional imaging data 122 may be imaging data for a baseline point in time for the subject prior to treatment or concurrent with a first dose of a treatment.

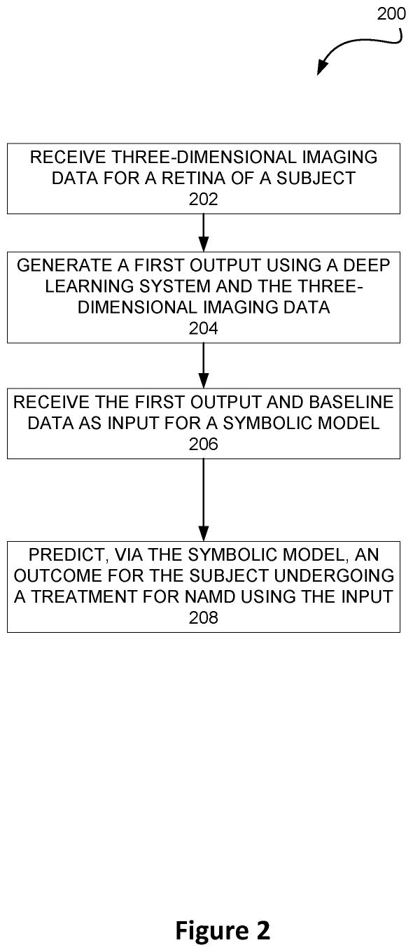

Model system 112 processes set of inputs 110 to predict at least one treatment outcome 124 for a subject who has or will undergo an nAMD treatment. Treatment outcome 124 may include, for example, without limitation, at least one of a predicted visual acuity measurement (e.g., a predicted BCVA), a predicted changed in visual acuity (e.g., a predicted change in BCVA), a predicted CST, a predicted reduction in CST, or some other type of treatment outcome of a subject undergoing treatment. Treatment outcome 124 may be generated for a selected point in time after a baseline point in time. For example, treatment outcome 124 may be predicted at an n^{th} month after a baseline point in time, the nth month being selected as a month between three months and thirty months after the baseline point in time. In one or more embodiments, treatment outcome 124 may be predicted for a time such as, without limitation, 6 months, 9 months, 12 months, 24 months, or some other amount of time after treatment. Examples of how model system 112 can be used to predict treatment outcome 124 are described in greater detail in FIGS. 2-4 below.

Data analyzer 108 may use treatment outcome 124 to form outcome output 114. Outcome output 114 may include, for example, treatment outcome 124. In one or more embodiments, outcome output 114 includes multiple treatment outcomes for multiple points in time after treatment (e.g., a treatment outcome for 6 months, a treatment outcome for 9 months, and a treatment outcome for 12 months).

In one or more embodiments, outcome output 114 includes other information generated based on treatment outcome 124. For example, outcome output 114 may include a personalized treatment regimen for a given subject based on the predicted treatment outcome 124. In some examples, outcome output 114 may include a customized injection dosage, one or more intervals at which injections are to be given, or both. Outcome output 114 may include, in some cases, an indication to change or supplement the type of treatment to be administered to the subject based on the predicted treatment outcome 124 indicating that the subject will not have a desired response to the treatment. In this manner, outcome output 114 may be used to improve overall treatment management.

In one or more embodiments, at least a portion of outcome output 114 or a graphical representation of at least a portion of outcome output 114 is displayed on display system 106. In some embodiments, at least a portion of outcome output 114 or a graphical representation of at least a portion of outcome output 114 is sent to remote device 126 (e.g., a mobile device, a laptop, a server, a cloud, etc.).

II.B. Exemplary Methodologies for Predicting AMD Treatment Outcomes

FIG. 2 is a flowchart of a process 200 for predicting a treatment outcome in accordance with various embodiments. In one or more embodiments, process 200 is implemented using prediction system 100 described in FIG. 1.

Step 202 includes receiving three-dimensional imaging data for a retina of a subject. Three-dimensional imaging data 122 in FIG. 1 may be one example of an implementation for the three-dimensional imaging data in step 202. The three-dimensional imaging data may include OCT imaging data, data extracted from OCT images (e.g., OCT en-face images), tabular data extracted from OCT images, some other form of imaging data, or a combination thereof. The OCT imaging data may include, for example, spectral domain OCT (SD-OCT) B-scans. The three dimensional imaging data may be imaging data for a baseline point in time for the subject prior to treatment or concurrent with a first dose of a treatment.

Step 204 includes generating a first output using a deep learning system and the three-dimensional imaging data. First outcome predictor model 116 described in FIG. 1 may be one example of an implementation for the deep learning system used in step 204. The deep learning system may be comprised of one or more neural networks. In one or more embodiments, the first output generated in step 204 is a predicted outcome (e.g., a predicted treatment outcome). For example, the deep learning system may have been trained to predict a treatment outcome based on one or more OCT images generated at a baseline point in time for the subject.

Step 206 includes receiving the first output and baseline data as input for a symbolic model. Second outcome predictor model 118 described in FIG. 1 may be one example of an implementation for the symbolic model used in step 206. The symbolic model may be implemented using, for example, at least one of a linear model, a random forest model, an XGBoost algorithm, or another type of symbolic learning model. Baseline data 120 in FIG. 1 may be one example of an implementation for the baseline data in step 206. The baseline data may include, for example, at least one of demographic data (e.g., age, gender, etc.), a baseline visual acuity measurement (e.g., a baseline BCVA), a baseline central subfield thickness (CST) measurement, a baseline low-luminance deficit (LLD), or a treatment arm.

Step 208 includes predicting (or generating), via the symbolic model, a treatment outcome for a subject undergoing a treatment for neovascular age-related macular degeneration (nAMD) using the input. The treatment outcome may include, for example, without limitation, at least one of a predicted visual acuity measurement (e.g., a predicted BCVA), a predicted change in visual acuity, a predicted CST, a predicted reduction in CST, or another indicator of the response of a subject to the treatment. The treatment outcome predicted in step 208 may be for a selected point in time after treatment such as, for example, without limitation, 6 months, 9 months, 12 months, 24 months, or some other amount of time after treatment.

In various embodiments, the treatment outcome predicted (or generated) in step 208 includes a visual acuity response (VAR) output that is a value or score that identifies the predicted change in the visual acuity of the subject. For example, the VAR output may be a value or score that classifies the subject's visual acuity response with respect to the level of improvement predicted (e.g., letters of improvement) or decline (e.g., vision loss). As one specific example, the VAR output may be a predicted numeric change in BCVA that is later processed and identified as belonging to one of a plurality of different classes of BCVA change, each class of BCVA change corresponding to a different range of letters of improvement. As another example, the VAR output may be the predicted class of change itself. In still other examples, the VAR output may be a predicted change in some other measure of visual acuity. In other embodiments, the VAR output may be a value or representational output that requires one or more additional processing steps to arrive at the predicted change in visual acuity. For example, the VAR output may be a predicted, future BCVA of the subject at a period of time post-treatment (e.g., at 9 months, at 12 months). The additional one or more processing steps may include computing the difference between the predicted, future BCVA and the baseline BCVA to determine the predicted change in visual acuity.

Process 200 may optionally include step 210. Step 210 includes generating an outcome output based on the treatment outcome. Outcome output 114 in FIG. 1 may be one example of an implementation for the outcome output in step 210. The outcome output may include, for example, the treatment outcome or multiple treatment outcomes for multiple points in time after treatment (e.g., a treatment outcome for 6 months, a treatment outcome for 9 months, and a treatment outcome for 12 months).

In one or more embodiments, the outcome output includes other information generated based on the treatment outcome. For example, the outcome output may include a personalized treatment regimen for a given subject based on the predicted treatment outcome. In some examples, the outcome output may include a customized injection dosage, one or more intervals at which injections are to be given, or both. The outcome output may include, in some cases, an indication to change or supplement the type of treatment to be administered to the subject based on the predicted treatment outcome indicating that the subject will not have a desired response to the treatment. In this manner, the outcome output may be used to improve overall treatment management.

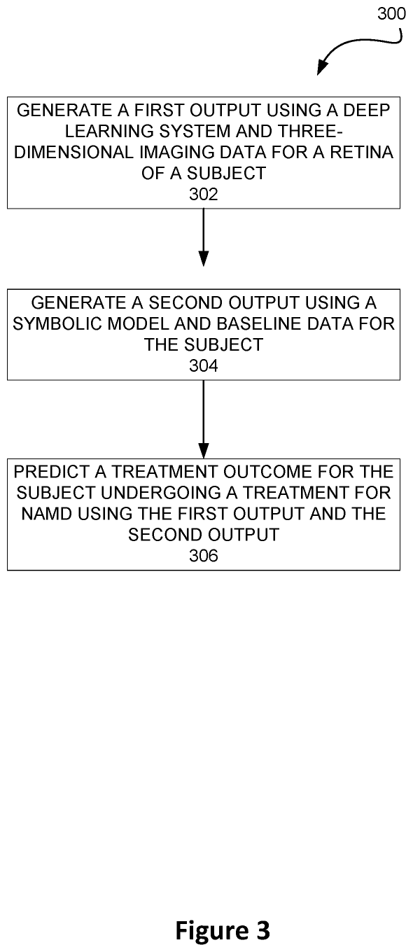

FIG. 3 is a flowchart of a process 300 for predicting a treatment outcome in accordance with various embodiments. In one or more embodiments, process 300 is implemented using prediction system 100 described in FIG. 1.

Step 302 includes generating a first output using a deep learning system and three-dimensional imaging data of a retina of a subject. Three-dimensional imaging data 122 in FIG. 1 may be one example of an implementation for the three-dimensional imaging data in step 302. The three-dimensional imaging data may include OCT imaging data, data extracted from OCT images (e.g., OCT en-face images), tabular data extracted from OCT images, some other form of imaging data, or a combination thereof. The OCT imaging data may include, for example, spectral domain OCT (SD-OCT) B-scans. The three dimensional imaging data may be imaging data for a baseline point in time for the subject prior to treatment or concurrent with a first dose of a treatment.

The first output in step 302 may include a first predicted outcome (a first predicted treatment outcome). For example, the deep learning system may be trained to generate the first predicted outcome based on the three-dimensional imaging data.

Step 304 includes generating a second output using a symbolic model and baseline data. Baseline data 120 in FIG. 1 may be one example of an implementation for the baseline data in step 304. The baseline data may include, for example, at least one of demographic data (e.g., age, gender, etc.), a baseline visual acuity measurement (e.g., a baseline BCVA), a baseline central subfield thickness (CST) measurement, a baseline low-luminance deficit (LLD), or a treatment arm.

The second output in step 304 may include a second predicted outcome (a second predicted treatment outcome). For example, the symbolic model may be trained to generate the second predicted outcome based on the baseline data. Second outcome predictor model 118 described in FIG. 1 may be one example of an implementation for the symbolic model used in step 304. The symbolic model may be implemented using, for example, at least one of a linear model, a random forest model, an XGBoost algorithm, or another type of symbolic learning model.

Step 306 includes predicting the treatment outcome for the subject undergoing the treatment for nAMD using the first output and the second output. In one or more embodiments, step 306 includes predicting the treatment outcome as a weighted average (e.g., equally weighted average) of the first output (e.g., the first predicted outcome) and the second output (e.g., the second predicted outcome). In some embodiments, the first predicted outcome generated by the deep learning system may be weighted greater than the second predicted outcome generated by the symbolic model. In other embodiments, the second predicted outcome generated by the symbolic model may be weighted greater than the first predicted outcome generated by the deep learning system.

Process 300 may optionally include step 308. Step 308 may include generating an outcome output based on the treatment outcome. Outcome output 114 in FIG. 1 may be one example of an implementation for the outcome output in step 308. The outcome output may include, for example, the treatment outcome or multiple treatment outcomes for multiple points in time after treatment (e.g., a treatment outcome for 6 months, a treatment outcome for 9 months, and a treatment outcome for 12 months).

In one or more embodiments, the outcome output includes other information generated based on the treatment outcome. For example, the outcome output may include a personalized treatment regimen for a given subject based on the predicted treatment outcome. In some examples, the outcome output may include a customized injection dosage, one or more intervals at which injections are to be given, or both. The outcome output may include, in some cases, an indication to change or supplement the type of treatment to be administered to the subject based on the predicted treatment outcome indicating that the subject will not have a desired response to the treatment. In this manner, the outcome output may be used to improve overall treatment management.

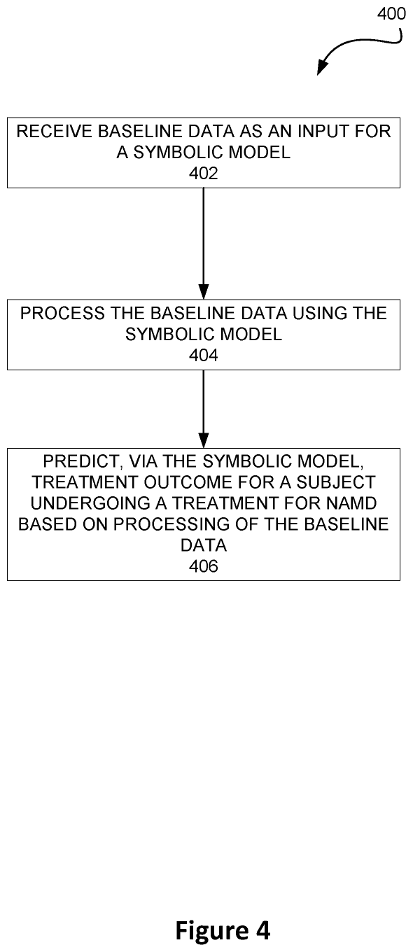

FIG. 4 is a flowchart of a process 400 for predicting a treatment outcome in accordance with various embodiments. In one or more embodiments, process 400 is implemented using prediction system 100 described in FIG. 1.

Step 402 includes receiving baseline data as an input for a symbolic model. Baseline data 120 in FIG. 1 may be one example of an implementation for the baseline data in step 402. Further, second outcome predictor model 118 described in FIG. 1 may be one example of an implementation for the symbolic model used in step 206. The symbolic model may be implemented using, for example, at least one of a linear model, a random forest model, an XGBoost algorithm, or another type of symbolic learning model. In one or more embodiments, the baseline data includes a baseline visual acuity measurement (e.g., a baseline BCVA). This baseline visual acuity measurement may have been generated using three-dimensional imaging data (e.g., OCT imaging data) and a deep learning system.

Step 404 includes processing the baseline data using the symbolic model. The symbolic model may use any number of symbolic artificial intelligence learning methodologies to process the baseline data. In some embodiments, step 404 includes processing the baseline data and a previously generated treatment outcome received from another system (e.g., a deep learning system).

Step 406 includes predicting, via the symbolic model, a treatment outcome for a subject undergoing a treatment for nAMD based on the processing of the baseline data. Treatment outcome 124 in FIG. 1 may be one example of an implementation for the treatment outcome.

### III. Exemplary Experimental Data

A first study was conducted using data from 185 eyes treated with an nAMD treatment (e.g., faricimab). This data was obtained for subjects from the AVENUE clinical trial (NCT02484690) who were randomized into four faricimab treatment arms. The data for a particular eye included baseline data and post-treatment data. The baseline data included demographic data (age, gender), a baseline BCVA, a baseline CST, a low-luminance deficit, and treatment arm. The data further included SD-OCT imaging data (e.g., B scans) of the eyes. The post-treatment data included complete BCVA data, CST at month 9 after treatment. The data was split into 80% training data and 20% testing data.

Treatment outcomes were predicted using a deep learning system (e.g., an example of an implementation for first outcome predictor model 116 in FIG. 1) and various symbolic models (e.g., examples of implementations for second outcome predictor model 118 in FIG. 1). Treatment outcomes were defined in two ways: functional and anatomical. The functional portion of a treatment outcome included a VAR output (e.g., a BCVA letter score at month 9). The anatomical portion of the treatment outcome included a CST reduction rate from the baseline point in time to month 9, with the CST reduction rate being converted into a binary true/false variable (e.g., with true indicating a CST reduction rate greater than 35%). The threshold (e.g., 35%) for the binary variable was selected based on an average or median CST reduction rate for the subjects.

The primary metric for the functional portion of the treatment outcome was a coefficient of determination (R²) score. The primary metric for the anatomical portion of the treatment outcome was an area under the receiver operator characteristic (AUROC) curve. Secondary metrics included accuracy, precision, and recall. Evaluation of model performance included 5-fold cross validation.

In a model stacking approach comprising two stages involving a given symbolic model, the deep learning system was first used to generate a predicted outcome in a first stage. This predicted outcome was then used as one of the input features, along with the baseline data, for the symbolic model in a second stage. 5-fold cross validation was used to tune hyper-parameters of the deep learning system and the symbolic model. For the first stage, 5-fold CV was used to tune hyper-parameters of the deep learning system. In iteration i (i=1, 2, 3, 4, 5) of the second stage 5-fold cross validation, the prediction of the deep learning system from iteration i of the first stage 5-fold cross validation was used as one of the input features in combination with the baseline data. Six total models were developed using the model stacking approach.

In a model averaging approach, for a given symbolic model, the predicted outcome generated by the deep learning system and the predicted outcome generated by the symbolic model were averaged together (e.g., via equal weighting) to generate the predicted treatment outcome. Six total models were developed using the model averaging approach.

To calculate test data performance metrics, the symbolic model was retrained on the entire training data set with the optimal hyper-parameters found in 5-fold cross validation. The deep learning system was used in an ensemble way, that is, the average of the five deep learning systems (i.e., from each 5-fold CV iteration) was used.

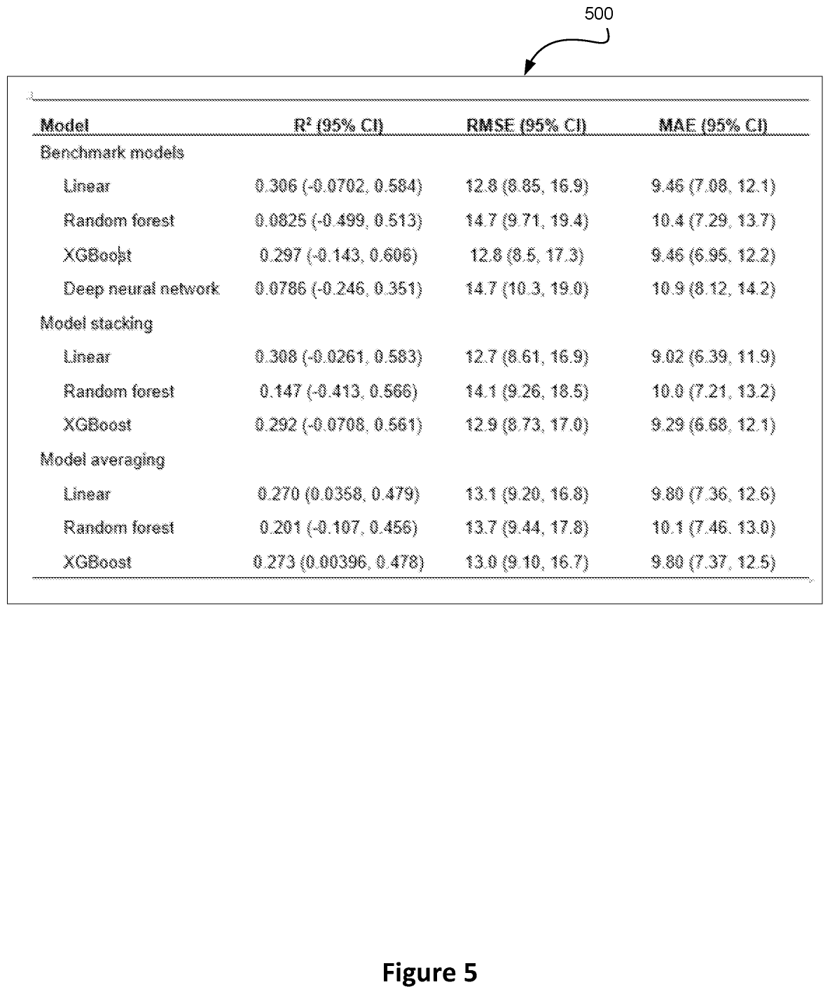

FIG. 5 is a table showing the performance data for a model stacking and model averaging approach in predicting a treatment outcome in accordance with one or more embodiments. The treatment outcome includes a predicted BCVA at month 9. The benchmark models identify each individual model that was used. With respect to model stacking, the identified model is the symbolic model that was stacked with the deep learning system. With respect to model averaging, the identified model is the symbolic model whose output was averaged with the output of the deep learning system.

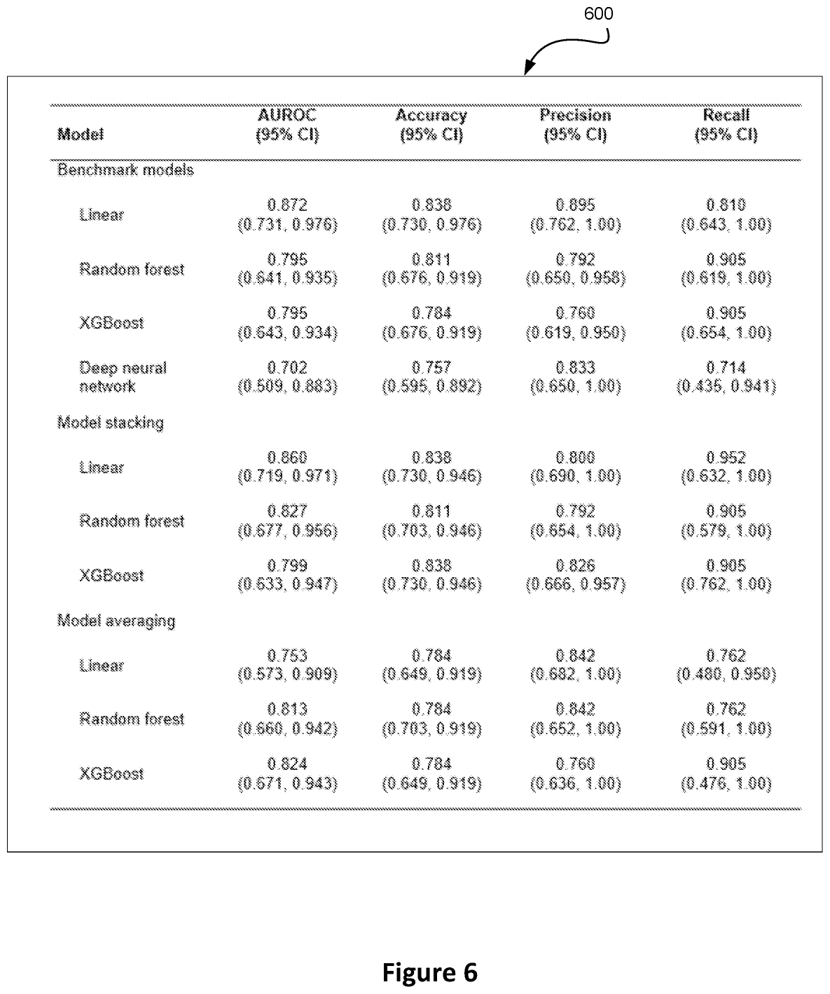

FIG. 6 is a table showing the performance data for a model stacking and model averaging approach in predicting a treatment outcome in accordance with one or more embodiments. The treatment outcome includes a CST reduction rate classification where a true or positive classification indicates a CST reduction rate of greater than 35%. The benchmark models identify each individual model that was used. With respect to model stacking, the identified model is the symbolic model that was stacked with the deep learning system. With respect to model averaging, the identified model is the symbolic model whose output was averaged with the output of the deep learning system.

### IV. Computer-Implemented System

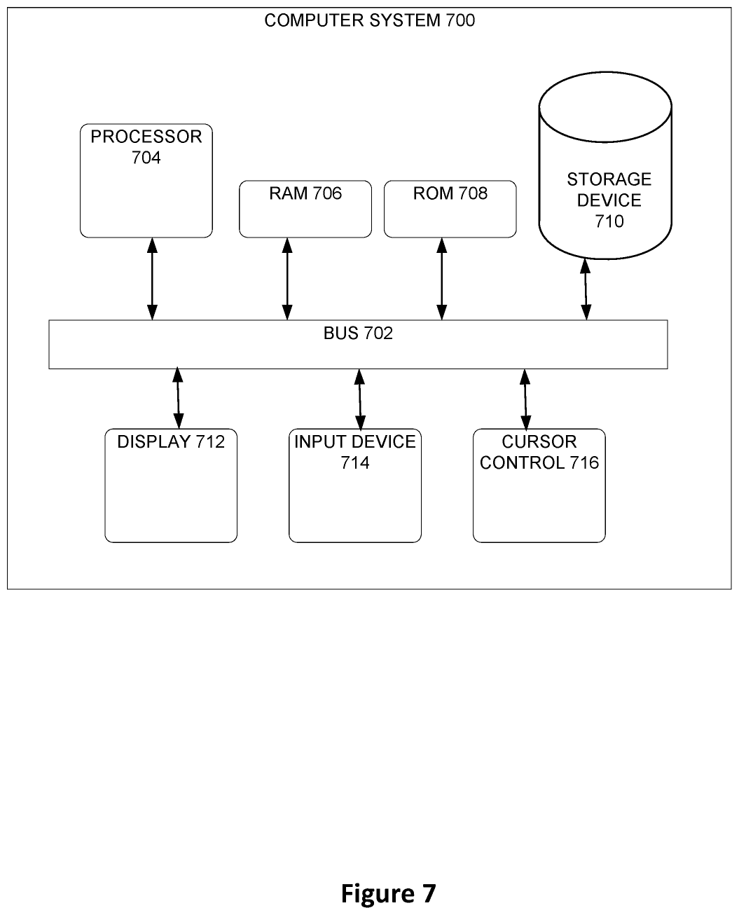

FIG. 7 is a block diagram illustrating an example of a computer system in accordance with various embodiments. Computer system 700 may be an example of one implementation for computing platform 102 described above in FIG. 1. In one or more examples, computer system 700 can include a bus 702 or other communication mechanism for communicating information, and a processor 704 coupled with bus 702 for processing information. In various embodiments, computer system 700 can also include a memory, which can be a random-access memory (RAM) 706 or other dynamic storage device, coupled to bus 702 for determining instructions to be executed by processor 704. Memory also can be used for storing temporary variables or other intermediate information during execution of instructions to be executed by processor 704. In various embodiments, computer system 700 can further include a read-only memory (ROM) 708 or other static storage device coupled to bus 702 for storing static information and instructions for processor 704. A storage device 710, such as a magnetic disk or optical disk, can be provided and coupled to bus 702 for storing information and instructions.

In various embodiments, computer system 700 can be coupled via bus 702 to a display 712, such as a cathode ray tube (CRT) or liquid crystal display (LCD), for displaying information to a computer user. An input device 714, including alphanumeric and other keys, can be coupled to bus 702 for communicating information and command selections to processor 704. Another type of user input device is a cursor control 716, such as a mouse, a joystick, a trackball, a gesture-input device, a gaze-based input device, or cursor direction keys for communicating direction information and command selections to processor 704 and for controlling cursor movement on display 712. This input device 714 typically has two degrees of freedom in two axes, a first axis (e.g., x) and a second axis (e.g., y), that allows the device to specify positions in a plane. However, it should be understood that input devices 714 allowing for three-dimensional (e.g., x, y and z) cursor movement are also contemplated herein.

Consistent with certain implementations of the present teachings, results can be provided by computer system 700 in response to processor 704 executing one or more sequences of one or more instructions contained in RAM 706. Such instructions can be read into RAM 706 from another computer-readable medium or computer-readable storage medium, such as storage device 710. Execution of the sequences of instructions contained in RAM 706 can cause processor 704 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 704 for execution. Such a medium can take many forms, including but not limited to, non-volatile media, volatile media, and transmission media. Examples of non-volatile media can include, but are not limited to, optical, solid state, magnetic disks, such as storage device 710. Examples of volatile media can include, but are not limited to, dynamic memory, such as RAM 706. Examples of transmission media can include, but are not limited to, coaxial cables, copper wire, and fiber optics, including the wires that comprise bus 702.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 704 of computer system 700 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 700 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 700, whereby processor 704 would execute the analyses and determinations provided by these engines, subject to instructions provided by any one of, or a combination of, the memory components RAM 706, ROM, 708, or storage device 710 and user input provided via input device 714.

### V. Exemplary Definitions and Context

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. In various cases, “subject” and “patient” may be used interchangeably herein.

Unless otherwise defined, scientific and technical terms used in connection with the present teachings described herein shall have the meanings that are commonly understood by those of ordinary skill in the art. Further, unless otherwise indicated by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” may mean within ten percent.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and, in some cases, only one of the items in the list may be used. The item may be a particular object, thing, step, operation, process, or category. In other words, “at least one of” means any combination of items or number of items may be used from the list, but not all of the items in the list may be used. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming.

As used herein, an “artificial neural network” or “neural network” (NN) may refer to mathematical algorithms or computational models that mimic an interconnected group of artificial neurons that processes information based on a connectionistic approach to computation. Neural networks, which may also be referred to as neural nets, can employ one or more layers of linear units, nonlinear units, or both to predict an output for a received input. Some neural networks include one or more hidden layers in addition to an output layer. The output of each hidden layer may be used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways. For example, it may process information when it is being trained in training mode and when it puts what it has learned into practice in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may learn by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

### VI. Recitation of Embodiments

Embodiment 1. A method for predicting a treatment outcome, the method comprising: receiving three-dimensional imaging data for a retina of a subject; generating a first output using a deep learning system and the three-dimensional imaging data; receiving the first output and baseline data as input for a symbolic model; and predicting, via the symbolic model, a treatment outcome for the subject undergoing a treatment for neovascular age-related macular degeneration (nAMD) using the input.

Embodiment 2. The method of embodiment 1, wherein the three-dimensional imaging data comprises optical coherence tomography (OCT) imaging data.

Embodiment 3. The method of embodiment 1 or embodiment 2, wherein the baseline data comprises at least one of demographic data, a baseline visual acuity measurement, a baseline central subfield thickness measurement, a baseline low-luminance deficit, or a treatment arm.

Embodiment 4. The method of embodiment 3, wherein the demographic data comprises at least one of age or gender.

Embodiment 5. The method of any one of embodiments 1-4, wherein the treatment outcome includes at least one of a predicted visual acuity measurement, a predicted change in visual acuity, a predicted central subfield thickness, or a predicted reduction in central subfield thickness.

Embodiment 6. The method of any one of embodiments 1-5, wherein the baseline data includes a baseline visual acuity measurement and further comprising:
identifying the baseline visual acuity measurement using the first output.

Embodiment 7. The method of any one of embodiments 1-6, wherein the treatment outcome is predicted at an n^{th} month after a baseline point in time and wherein the n^{th} month is selected as a month between three months and thirty months after the baseline point in time.

Embodiment 8. The method of any one of embodiments 1-7, wherein the treatment comprises a monoclonal antibody that targets vascular endothelial growth factor, and angiopoietin 2 inhibitor.

Embodiment 9. The method of any one of embodiments 1-8, wherein the treatment comprises faricimab.

Embodiment 10. A method for predicting a treatment outcome for a subject undergoing a treatment for neovascular age-related macular degeneration (nAMD), the method comprising: generating a first predicted outcome using a deep learning system and three-dimensional imaging data for a retina of the subject; generating a second predicted outcome using a symbolic model and baseline data for the subject; and predicting the treatment outcome for the subject undergoing the treatment for nAMD using the first predicted outcome and the second predicted outcome.

Embodiment 11. The method of embodiment 10, wherein the predicting comprises: predicting the treatment outcome as a weighted average of the first predicted treatment outcome and the second predicted treatment outcome.

Embodiment 12. The method of embodiment 10 or embodiment 11, wherein the three-dimensional imaging data comprises optical coherence tomography (OCT) imaging data.

Embodiment 13. The method of any one of embodiments 10-12, wherein the baseline data comprises at least one of demographic data, a baseline visual acuity measurement, a baseline central subfield thickness measurement, a baseline low-luminance deficit, or a treatment arm.

Embodiment 14. The method of embodiment 13, wherein the demographic data comprises at least one of age or gender.

Embodiment 15. The method of any one of embodiments 10-14, wherein each of the first predicted treatment outcome, the second predicted treatment outcome, and the treatment outcome includes at least one of a predicted visual acuity measurement, a predicted change in visual acuity, a predicted central subfield thickness, or a predicted reduction in central subfield thickness.

Embodiment 16. A system for managing an anti-vascular endothelial growth factor (anti-VEGF) treatment for a subject diagnosed with neovascular age-related macular degeneration (nAMD), the system comprising: a memory containing machine readable medium comprising machine executable code; and a processor coupled to the memory, the processor configured to execute the machine executable code to cause the processor to:
receive three-dimensional imaging data for a retina of a subject; generate a first output using a deep learning system and the three-dimensional imaging data;
receive the first output and baseline data as input for a symbolic model; and
predict, via the symbolic model, a treatment outcome for the subject undergoing a treatment for neovascular age-related macular degeneration (nAMD) using the input.

Embodiment 17. The system of embodiment 16, wherein the three-dimensional imaging data comprises optical coherence tomography (OCT) imaging data.

Embodiment 18. The system of embodiment 16 or embodiment 17, wherein the baseline data comprises at least one of demographic data, a baseline visual acuity measurement, a baseline central subfield thickness measurement, a baseline low-luminance deficit, or a treatment arm.

Embodiment 19. The system of any one of embodiments 16-18, wherein the treatment outcome includes at least one of a predicted visual acuity measurement, a predicted change in visual acuity, a predicted central subfield thickness, or a predicted reduction in central subfield thickness.

Embodiment 20. The system of any one of embodiments 16-18, wherein the treatment comprises faricimab.

Embodiment 21. A method for predicting a treatment outcome, the method comprising: receiving baseline data as an input for a symbolic model; processing the baseline data using the symbolic model; and predicting, via the symbolic model, an outcome for a subject undergoing a treatment based on the processing of the baseline data.

Embodiment 22. The method of embodiment 21, wherein the baseline data includes a baseline visual acuity measurement and further comprising: generating the baseline visual acuity measurement using three-dimensional imaging data and a deep learning system.

### VII. Additional Considerations

The headers and subheaders between sections and subsections of this document are included solely for the purpose of improving readability and do not imply that features cannot be combined across sections and subsection. Accordingly, sections and subsections do not describe separate embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

While the present teachings are described in conjunction with various embodiments, it is not intended that the present teachings be limited to such embodiments. On the contrary, the present teachings encompass various alternatives, modifications, and equivalents, as will be appreciated by those of skill in the art. In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments. It is understood that various changes may be made in the function and arrangement of elements (e.g., elements in block or schematic diagrams, elements in flow diagrams, etc.) without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A method for predicting a treatment outcome, the method comprising:
receiving three-dimensional imaging data for a retina of a subject;
generating a first output using a deep learning system and the three-dimensional imaging data;
receiving the first output and baseline data as input for a symbolic model; and
predicting, via the symbolic model, a treatment outcome for the subject undergoing a treatment for neovascular age-related macular degeneration (nAMD) using the input.

2. The method of claim 1, wherein the three-dimensional imaging data comprises optical coherence tomography (OCT) imaging data.

3. The method of claim 1 or claim 2, wherein the baseline data comprises at least one of demographic data, a baseline visual acuity measurement, a baseline central subfield thickness measurement, a baseline low-luminance deficit, or a treatment arm.

4. The method of claim 3, wherein the demographic data comprises at least one of age or gender.

5. The method of any one of claims 1-4, wherein the treatment outcome includes at least one of a predicted visual acuity measurement, a predicted change in visual acuity, a predicted central subfield thickness, or a predicted reduction in central subfield thickness.

6. The method of any one of claims 1-5, wherein the baseline data includes a baseline visual acuity measurement and further comprising:
identifying the baseline visual acuity measurement using the first output.

7. The method of any one of claims 1-6, wherein the treatment outcome is predicted at an n th month after a baseline point in time and wherein the n^{th} month is selected as a month between three months and thirty months after the baseline point in time.

8. The method of any one of claims 1-7, wherein the treatment comprises a monoclonal antibody that targets vascular endothelial growth factor, and angiopoietin 2 inhibitor.

9. The method of any one of claims 1-8, wherein the treatment comprises faricimab.

10. A method for predicting a treatment outcome for a subject undergoing a treatment for neovascular age-related macular degeneration (nAMD), the method comprising:
generating a first predicted outcome using a deep learning system and three-dimensional imaging data for a retina of the subject;
generating a second predicted outcome using a symbolic model and baseline data for the subject; and
predicting the treatment outcome for the subject undergoing the treatment for nAMD using the first predicted outcome and the second predicted outcome.

11. The method of claim 10, wherein the predicting comprises:
predicting the treatment outcome as a weighted average of the first predicted treatment outcome and the second predicted treatment outcome.

12. The method of claim 10 or claim 11, wherein the three-dimensional imaging data comprises optical coherence tomography (OCT) imaging data.

13. The method of any one of claims 10-12, wherein the baseline data comprises at least one of demographic data, a baseline visual acuity measurement, a baseline central subfield thickness measurement, a baseline low-luminance deficit, or a treatment arm.

14. The method of claim 13, wherein the demographic data comprises at least one of age or gender.

15. The method of any one of claims 10-14, wherein each of the first predicted treatment outcome, the second predicted treatment outcome, and the treatment outcome includes at least one of a predicted visual acuity measurement, a predicted change in visual acuity, a predicted central subfield thickness, or a predicted reduction in central subfield thickness.

16. A system for managing an anti-vascular endothelial growth factor (anti-VEGF) treatment for a subject diagnosed with neovascular age-related macular degeneration (nAMD), the system comprising:
a memory containing machine readable medium comprising machine executable code; and
a processor coupled to the memory, the processor configured to execute the machine executable code to cause the processor to:
receive three-dimensional imaging data for a retina of a subject;
generate a first output using a deep learning system and the three-dimensional imaging data;
receive the first output and baseline data as input for a symbolic model; and
predict, via the symbolic model, a treatment outcome for the subject undergoing a treatment for neovascular age-related macular degeneration (nAMD) using the input.

17. The system of claim 16, wherein the three-dimensional imaging data comprises optical coherence tomography (OCT) imaging data.

18. The system of claim 16 or claim 17, wherein the baseline data comprises at least one of demographic data, a baseline visual acuity measurement, a baseline central subfield thickness measurement, a baseline low-luminance deficit, or a treatment arm.

19. The system of any one of claims 16-18, wherein the treatment outcome includes at least one of a predicted visual acuity measurement, a predicted change in visual acuity, a predicted central subfield thickness, or a predicted reduction in central subfield thickness.

20. The system of any one of claims 16-18, wherein the treatment comprises faricimab.
